(19) United States
(12) Patent Application Publication
(10) Pub. No.: US 2022/0168505 A1
(43) Pub. Date: Jun. 2, 2022

(54) DEVICE AND METHODS FOR A SIMPLE MEAL ANNOUNCEMENT FOR AUTOMATIC DRUG DELIVERY SYSTEM

(71) Applicant: Insulet Corporation, Acton, MA (US)

(72) Inventors: Mengdi LI, Westford, MA (US); Yibin ZHENG, Harland, WI (US); Joon Bok LEE, Acton, MA (US); Jason O'CONNOR, Acton, MA (US)

(21) Appl. No.: 17/534,129

(22) Filed: Nov. 23, 2021

Related U.S. Application Data
(60) Provisional application No. 63/119,055, filed on Nov. 30, 2020.

(51) Int. Cl. A61M 5/172

(52) U.S. Cl. CPC A61M 5/1723; A61M 2005/14208; A61M 2230/201

(57) ABSTRACT

Processes and devices are disclosed that are configured to respond to changes in a user's blood glucose caused by ingestion of a meal. Ingestion of the meal may be announced by a user input or by a meal detection algorithm that requires no user input. The responsive device and processes determine a carbohydrate-compensation insulin dosage based on a user's blood glucose history, external data related to the user's meal history, or based on a user's response to previous carbohydrate-compensation insulin dosages. In addition, a correction insulin dosage may be calculated to cover any gap between a starting blood glucose and a target blood glucose. A user's response to a sum of the carbohydrate-compensation insulin dosage and the correction insulin dosage may be delivered. Based on the user's response, the disclosed examples may determine modifications to the carbohydrate-compensation insulin dosage, the correction insulin dosage, or both.

### RELATED APPLICATIONS

This application claims the benefit of U.S. Provisional Patent Application No. 63/119,055, filed Nov. 30, 2020, the contents of which are incorporated herein by reference in their entirety.

### BACKGROUND

Currently, some of the state of the art meal bolus calculator requires users to input their estimated carbohydrate intake. The meal size and estimation error vary from person to person. The maximum estimation error can be around ±25 g.

Some hybrid automatic insulin delivery systems may require user to manually prescribe an insulin dose to compensate for meal or carbohydrate intakes. The manual prescription process involves users estimating the carbohydrate amount and using a bolus calculator, which is burdensome and prone to error for many less technical users.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

In some approaches, a method may include receiving a meal announcement. The meal announcement may be a notification of ingestion of a meal. In response to the announcement of ingestion of the meal, a carbohydrate-compensation dosage of insulin may be estimated. An amount of insulin-on-board (JOB) based on insulin delivery history may be estimated. A current blood glucose measurement value may be obtained. A correction insulin dosage may be estimated using the estimated amount of IOB and the current blood glucose measurement value. Upon completion of estimating a correction insulin dosage, a sum of the estimated carbohydrate-compensation dosage of insulin and the correction insulin dosage may be delivered. Changes in blood glucose measurement value over time may be monitored and basal insulin may be delivered to bring blood glucose measurement value into a set blood glucose measurement range. Within a preset time of receiving the meal announcement, a determination of whether a blood glucose measurement value obtained within the preset time has exceeded a hyperglycemia threshold or fallen below a hypoglycemia threshold may be made. In response to the blood glucose measurement value obtained within the preset time exceeding a hyperglycemia threshold or fallen below a hypoglycemia threshold, the carbohydrate-compensation dosage of insulin may be adapted by a predetermined factor.

In other approaches, another method may include obtaining a user's total daily insulin, a user's target blood glucose and a user's current blood glucose measurement. A carbohydrate-compensation insulin dosage may be estimated using the obtained total daily insulin. A correction insulin dosage may be estimated using the user's target blood glucose and the user's blood glucose measurement. The carbohydrate-compensation insulin dosage and the correction insulin dosage may be combined for a total bolus that is delivered. A status of a user's blood glucose and other information related to the user's blood glucose may be monitored. Based on a determination of the status of the user's blood glucose and other information related to the user's blood glucose, whether the total bolus underdelivered insulin may be determined. Based on the determination that the total bolus underdelivered insulin, a determination whether to update a carbohydrate-compensation estimation algorithm may be made. An update of a future carbohydrate-compensation insulin dosage may be generated based on a determination of the status of the user's blood glucose and other information related to the user's blood glucose.

In a further approach, a drug delivery device that includes a memory and a controller is provided. The memory may store programming code and the controller may be configured to execute the programming code. Execution of the programming code may configure the controller to receive a meal announcement, which is notification of ingestion of a meal. In response to the announcement of ingestion of the meal, a carbohydrate-compensation dosage of insulin may be estimated. Based on insulin delivery history, an amount of insulin-on-board (IOB) may be estimated. A current blood glucose measurement value may be obtained, and a correction insulin dosage may be estimated using the estimated amount of IOB and the current blood glucose measurement value. A sum of the estimated carbohydrate-compensation dosage of insulin and the correction insulin dosage upon completion of estimating a correction insulin dosage may be delivered. Changes in blood glucose measurement value may be monitored over time. Basal insulin may be delivered to bring blood glucose measurement values into a set blood glucose measurement range. Within a preset time of receiving the meal announcement, a determination of whether a blood glucose measurement value obtained within the preset time has exceeded a hyperglycemia threshold or fallen below a hypoglycemia threshold may be made. In response to the blood glucose measurement value obtained within the preset time exceeding a hyperglycemia threshold or fallen below a hypoglycemia threshold, the carbohydrate-compensation dosage of insulin may be adapted by a predetermined factor.

### BRIEF DESCRIPTION OF THE DRAWINGS

In the drawings, like reference characters generally refer to the same parts throughout the different views. In the following description, various embodiments of the present disclosure are described with reference to the following drawings, in which:

FIG. 1A shows a flow chart of an exemplary process for determining a dosage of a bolus injection in response to a meal announcement;

FIG. 1B illustrates a flow chart of an alternative exemplary process for responding to a meal announcement;

FIG. 2 illustrates an example of a subprocess usable in the example processes of FIGS. 1A and 1B;

FIG. 3A illustrates a process usable in the example processes of FIGS. 1A and 1B to estimate a correction insulin dosage that accounts for an amount of insulin on-board for the user;

FIGS. 3B-3D illustrate examples of different timelines for responding to delivery of a carbohydrate-compensation dosage of insulin or a correction bolus of insulin;

FIG. 4 illustrates an example of a process for determining a long-term update to a carbohydrate-compensation insulin dosage that is usable with the process examples described in FIGS. 1A and 1B; and

FIG. 5 illustrates a functional block diagram of an exemplary system suitable for implementing the example processes and techniques described herein.

FIG. 6 illustrates an example of a graphical user interface usable with the disclosed techniques and devices.

### DETAILED DESCRIPTION

Systems, devices, computer-readable medium and methods in accordance with the present disclosure will now be described more fully hereinafter with reference to the accompanying drawings, where one or more embodiments are shown. The systems, devices, and methods may be embodied in many different forms and are not to be construed as being limited to the embodiments set forth herein. Instead, these embodiments are provided so the disclosure will be thorough and complete, and will fully convey the scope of methods and devices to those skilled in the art. Each of the systems, devices, and methods disclosed herein provides one or more advantages over conventional systems, components, and methods.

Various examples provide a method, a system, a device and a computer-readable medium for responding to inputs provided by sensors, such as an analyte sensor, and users of an automatic drug delivery system. The various devices and sensors that may be used to implement some specific examples may also be used to implement different therapeutic regimens using different drugs than described in the specific examples.

In one example, the disclosed methods, system, devices or computer-readable medium may perform actions related to managing a user's blood glucose in response to ingestion of a meal by the user.

The disclosed examples provide techniques that may be used with any additional algorithms or computer applications that manage blood glucose levels and insulin therapy. These algorithms and computer applications may be collectively referred to as “medication delivery algorithms” or “medication delivery applications” and may be operable to deliver different categories of drugs (or medications), such as chemotherapy drugs, pain relief drugs, diabetes treatment drugs (e.g., insulin and/or glucagon), blood pressure medication, or the like.

A type of medication delivery algorithm (MDA) may include an “artificial pancreas” algorithm-based system, or more generally, an artificial pancreas (AP) application. For ease of discussion, the computer programs and computer applications that implement the medication delivery algorithms or applications may be referred to herein as an “AP application.” An AP application may be configured to provide automatic delivery of insulin based on a blood glucose sensor input, such as signals received from an analyte sensor, such as a continuous blood glucose monitor, or the like. In an example, the artificial pancreas (AP) application when executed by a processor may enable monitoring of a user's blood glucose measurement values, determine an appropriate level of insulin for the user based on the monitored glucose values (e.g., blood glucose concentrations or blood glucose measurement values) and other information, such as information related to, for example, carbohydrate intake, exercise times, meal times or the like, and take actions to maintain a user's blood glucose value within an appropriate range. A target blood glucose value of the particular user may alternatively be a range blood glucose measurement values that are appropriate for the particular user. For example, a target blood glucose measurement value may be acceptable if it falls within the range of 80 mg/dL to 120 mg/dL, which is a range satisfying the clinical standard of care for treatment of diabetes. In addition, an AP application as described herein may determine when a user's blood glucose wanders into the hypoglycemic range or the hyperglycemic range.

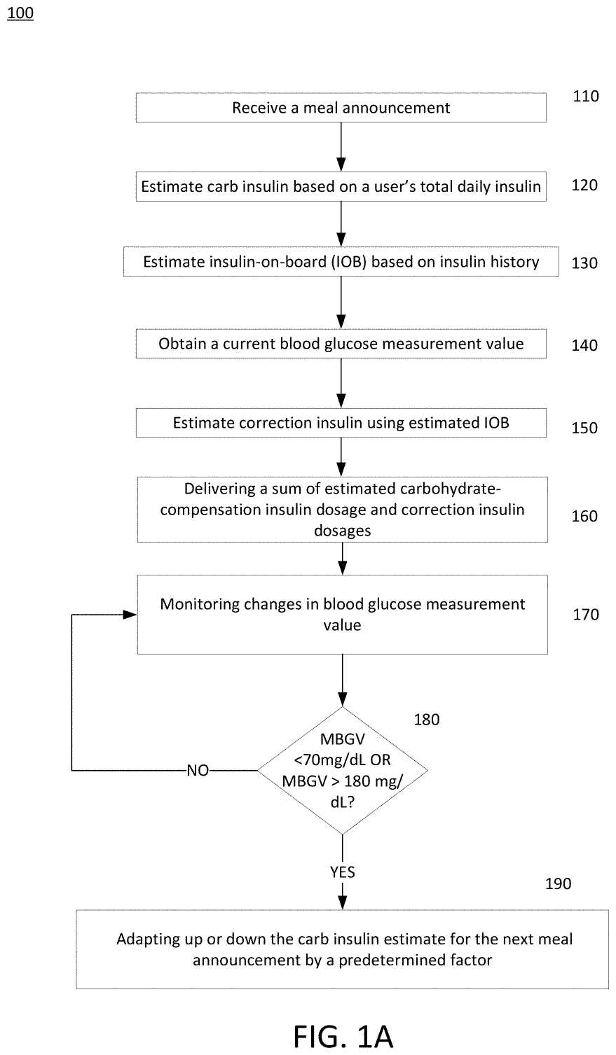

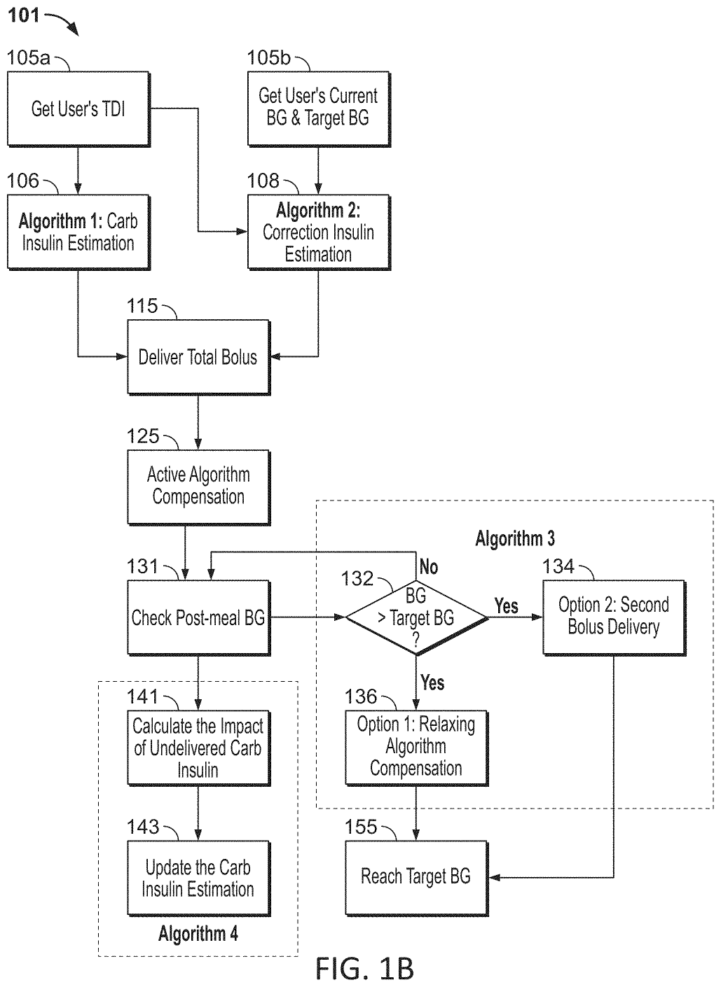

As described in more detail with reference to the examples of FIGS. 1A-4, an automatic drug delivery system may be configured to monitor a user's blood glucose measurement values, inputs from a user interface or a meal detection and response algorithm executed by a processor of a wearable automatic drug delivery device. The inputs from the user interface or the meal detection and response algorithm may be indications announcing that the user consumed or is about to consume a meal. The automatic drug delivery system may utilize the monitored information and/or the inputs to determine different dosages of medication to compensate for ingestion of the meal. The determined response to ingestion of the meal may be the determination of dosages of insulin that are intended to compensate for the increase in blood glucose that results from the carbohydrates in the consumed meal.

Typically, when responding to a meal, algorithms of the AP application without aid of the functions illustrated in the following examples implement a conservative approach due to uncertainty of the actual ingestion of a meal built into the respective meal detection algorithm. In contrast to this conservative approach, the disclosed examples may implement an aggressive delivery of insulin to more quickly yet appropriately compensate for consumption of a meal that adheres to reduced or decreased safety constraints. The following examples provide an AP application that is configured with a meal detection and response algorithm that is operable to modify post-prandial safety constraints that permit delivery of an amount of insulin to be administered to a user that more quickly compensates for consumption of the meal. As explained in more detail below, the examples of a meal detection and response algorithm may indicate the ingestion of a meal that enables the AP application to modify safety constraint settings for determination of the meal bolus, which enables more rapid compensation for meals.

An advantage of the disclosed examples is an automatic drug delivery (ADD) system enabled to determine that a meal has been consumed and modify safety constraints related to the consumed meal to enable the automatic insulin delivery system to administer an appropriate amount of insulin quickly and seamlessly without requiring a user to input details related to the consumed meal. Details related to the consumed meal may include identification of the composition of the meal (e.g., meat, starch, fruit, and the like), estimated number of carbohydrates and/or calories in the meal, meal size, estimated number of calories or carbohydrates, or the like. Using the described techniques, the system reduces the burden on the user when it is time to deliver insulin to compensate for changes in blood glucose measurement values as a result of consuming a meal and optimizes delivery of a correction bolus so a user may more quickly receive their bolus and begin lowering their blood glucose measurement value.

It may be helpful to describe in more detail the above examples as well as other examples of determining correction bolus dosages with reference to the drawings.

An advantage to be provided is simply allowing a user to provide an input that they are having a meal. Such an input may be a simple input to a soft button presented in a graphical user interface, a voice input to a control application, or the like. The meal announcement may cause the AP application to begin a process to compensate for ingestion of a meal.

Prior to the meal announcement, the AP application may have detected that the user's blood glucose was trending higher. For example, upon receipt of a blood glucose measurement value from an analyte sensor, which may be a blood glucose sensor, the blood glucose sensor may also provide an indication (e.g., a flag setting, a bit setting, or the like) of a direction of a trend, such as upward, downward or stable, of the blood glucose measurement value with respect to previously provided blood glucose measurement values The AP application may also compare the received blood glucose measurement value to a target blood glucose value and note when the target blood glucose value has been exceeded.

FIG. 1A shows a flow chart of an example process for determining a dosage of a bolus injection in response to a meal announcement. The example of the process illustrated in FIG. 1A may be implemented by an AP application executing on a processor. As shown in the process 100 example of FIG. 1A, the AP application may receive a meal announcement, at 110. The meal announcement may be a notification of ingestion of a meal provided by the user via a user input or an automated meal detection algorithm. For example, the meal announcement may be in response to a user engaging with a bolus button, a user verbally indicating a meal with a specific phrase, or a user shaking or otherwise physically interacting with the device. Alternatively, the AP application may utilize an automated meal detection algorithm that is configured to determine from one or more various inputs that a meal has been ingested. In either scenario, the AP application does not require the user to input an estimate of the carbohydrates in the meal.

In response to the meal announcement at 110, the AP application may obtain a user's total daily insulin (TDI). The TDI may, for example, be based on a weight of the user and/or the user's insulin delivery history.

In response to the meal announcement indicating ingestion of a meal, at 120 the AP application may estimate a carbohydrate-compensation dosage of insulin. The AP application may make the estimate using the user's carbohydrate history or the user's insulin delivery history. In addition, clustering algorithms may be used that are personalized to the user based on one or more of the user's carbohydrate history or the user's insulin delivery history or the like. In some examples, the carbohydrate-compensation dosage of insulin may be approximately 10 percent of a user's total daily insulin. At 130, the AP application may be configured to estimate an amount of insulin-on-board (JOB) for the user based on insulin delivery history of the user. At 140, a current blood glucose measurement value may be obtained from a blood glucose sensor or from a memory coupled to the processor. At 150, a correction insulin dosage may be estimated using the estimated amount of JOB. Upon completion of the estimate of the correction insulin dosage to be delivered, at 160 the AP application may be configured to cause a sum of the estimated carbohydrate-compensation dosage of insulin and the correction insulin dosage. The sum may be adjusted based on the user's starting blood glucose, IOB and a trend of the user's blood glucose measurement value. In an example, the AP application may output a control signal causing the delivery to occur immediately after the summation. As indicated at 170, changes in blood glucose measurement values may be monitored by the AP application over a period of time. The blood glucose measurement values over the period of time may change between 70 mg/dL and 180 mg/dL. The AP application may continue to deliver basal dosages of insulin as well as correction dosages of insulin to continue to bring blood glucose measurement values to a set range of the user's target blood glucose. At 180, the AP application may evaluate for a preset time period the monitored changes in blood glucose measurement values to determine whether the user's blood glucose has entered a hypoglycemic region (e.g., less than approximately 70 mg/dL) or a hyperglycemic region (e.g., greater than approximately 180 mg/dL). In response to determining the user's blood glucose has remained between the hypoglycemic region and the hyperglycemic region, the AP application may determine the result of the evaluation at 180 is “NO” and may continue to monitor the user's blood glucose at 170. Alternatively, if the determination is “YES” at 180, which means the determination that the blood glucose measurement value obtained within the preset time has exceeded a hyperglycemia threshold or fallen below a hypoglycemia threshold within a preset time period following the bolus, such as 5 hours, the AP application may respond by adapting the carbohydrate-compensation dosage of insulin by a predetermined factor. A predetermined factor may be between 5-10%, 10-15%, 5-15%, or the like.

In a further example, an updated correction insulin dosage may be estimated using an updated estimate of an amount of JOB. The processor may sum the adapted carbohydrate-compensation dosage of insulin and the updated correction insulin dosage and cause the sum of the adapted carbohydrate-compensation dosage of insulin and updated correction insulin dosage to be delivered upon completion of estimating correction insulin dosage.

FIG. 1B illustrates an alternative process example for responding to a meal announcement. Similar to process 100, the alternative process 101 does not require entry of any composition information (e.g., food items, portion size or the like), location information, carbohydrate information or any other nutritional information of the meal.

The process 101 may begin with the AP application obtaining the user's total daily insulin at 105a and obtaining a user's current blood glucose measurement and a target blood glucose at 105b. The steps 105a and 105b may occur sequentially or contemporaneously. At step 106, the carbohydrate-compensation insulin dosage may be estimated using the user's TDI obtained at 105a. At 108, the correction insulin dosage may be estimated utilizing the user's TDI obtained at 105a and the user's current blood glucose measurement and the target blood glucose obtained at 105b. Upon determining the carbohydrate-compensation insulin dosage at 106 and the correction insulin dosage at 108, the AP application may be operable to combine the carbohydrate-compensation insulin dosage and the correction insulin dosage for a total bolus. The total bolus may be delivered by a drug delivery device at 115.

After delivery of the total bolus, the AP application may continue to adapt settings of the AP application based on information received from the user, an analyte sensor, or the like. For example, the AP application may continue to actively monitor status of the user through receipt of blood glucose measurement values, blood glucose trend indicators, and other user-related metrics (such as, for example, calendar appointments, drug delivery device movement, or the like). At 125, the AP application may actively update and/or compensate the different parameters of the artificial pancreas algorithm that may affect the amount of insulin to be delivered based on the monitored status and user-related metrics.

The AP application may also continue to monitor the status of the user's blood glucose and other information, such as blood glucose trend, which is other information related to a user's blood glucose, insulin on board or a user's heart rate. Based on the user's blood glucose, and the other information that may include both blood glucose-related information (e.g., blood glucose trend or the like) and user's status information, such as heart rate, oxygen saturation or the like, the AP application may determine whether long-term actions, short-term actions, or both, need to be taken. For example, the AP application may obtain and check the post-meal blood glucose, at 131, and provide this information to two different subprocesses that respectively initiate long-term actions and short-term actions. A first subprocess to receive the results of the post-meal blood glucose check may be 141 that implements long-term actions (relative to the short-term actions). At 141, the algorithm is designed to estimate the impact of undelivered carb dosage of insulin, that may later be used to adjust the reduction proportion for a next meal. For example, for the previous meal, the carbohydrate-compensation bolus of insulin may be reduced by, for example, approximately 50% or 60% or the like and the post meal blood glucose measurement value may be much greater than a target blood glucose setting. The amount of decrease in the blood glucose measurement value resulting from the undelivered carbohydrate-compensation bolus dosage may be estimated. If the estimated blood glucose measurement value after subtracting the estimated decrease resulting from the undelivered carbohydrate-compensation bolus is close to the target blood glucose setting, the AP application may determine that it is safe to reduce the reduction proportion to, for example, approximately 40-50%, or more particularly, 45%, 48%, 50%, or the like, for a next meal. Based on the results of the determined impact of the undelivered carbohydrate-compensation insulin dosage, the AP application may update the algorithm for estimating the carbohydrate-compensation insulin dosage. For example, parameters or coefficients, such as insulin onboard (JOB), total daily insulin (TDI), target blood glucose setting, insulin sensitivity or the like, of the algorithm may be altered. For example, if a post meal blood glucose measurement value is still lower than 70 mg/dL, the AP application may consider and enable further increasing the reduction proportion for safety as well as increase the insulin sensitivity which serves to decrease the correction insulin delivery. The update to the algorithm for estimating the carbohydrate-compensation insulin dosage may be used to calculate an update of a future carbohydrate-compensation insulin dosage. The future carbohydrate-compensation insulin dosage may be used for a next meal, or may be used for a specific meal, such as, for example, breakfast or dinner.

A second subprocess that may implement short-term actions (relative to the longer-term actions of the process starting at 141) may be implemented, at 132, that may entail determining whether a blood glucose measurement value exceeds a target blood glucose setting. For example, the determination at 132 may determine whether the AP application is going to be more aggressive in its reduction of the user's blood glucose. In the event that the result at 132 is NO, the blood glucose measurement value does not exceed a target blood glucose setting, the process 101 returns to 131. However, should the result at 132 be “YES, the blood glucose measurement value exceeds a target blood glucose setting,” the process 101 may evaluate which of two options enable the user's blood glucose to reach the user's target blood glucose setting (136). For example, option 1 at 136 may be relaxing constraints on the algorithm for delivering insulin to compensate for consuming the meal. Alternative to option 1, option 2 at 134 may be, for example, implemented to determine whether a second bolus may be delivered and calculating the size of the second bolus, if it is determined the second bolus is to be delivered.

Depending upon which option is implemented, insulin may be delivered, or delivery of insulin may be delayed so the user's blood glucose reaches the user's target blood glucose setting, reflected at 155 in FIG. 1B.

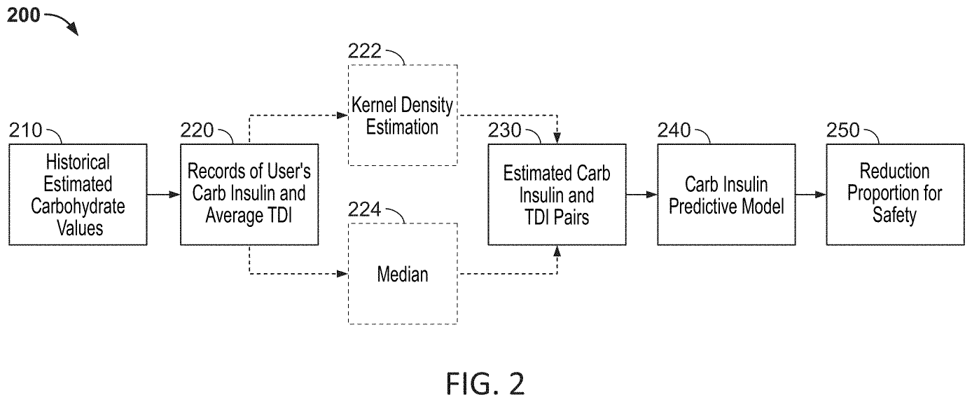

The processes 100 and 101 utilize subprocesses that enable determination of a carbohydrate-compensation insulin dosage. An example of such a subprocess is described with reference to FIG. 2. FIG. 2 illustrates an example of a subprocess usable in the example process of FIGS. 1A and 1B. In particular, the algorithm illustrated in FIG. 2 may be used to calculate an amount of insulin needed to compensate for a meal indicated by the meal announcement.

In the example of FIG. 2, the process 200 may enable an estimate of the carbohydrate-compensation dosage of insulin based on total daily insulin. For example, at 210, a processor may be configured to obtain a user's historical estimated carbohydrate values from a database, such as Glooko® data or the like. From the user's historical blood glucose measurement values, the process 200 may obtain the user's carbohydrate-compensation dosage and average total daily insulin at 220.

The processor may be further configured to build a linear regression model based on total daily insulin that may be used to predict a value for the user's carbohydrate-compensation insulin dosage. This prediction may be used when the user is a new user. Different methods may be used to make the prediction such as a kernel density estimation or the median value. Kernel density estimation is a process by which an estimate of the probability density function of a random variable may be made. The kernel density estimation derived carbohydrate-compensation insulin dosage and median derived carbohydrate-compensation insulin dosage may be based on the user's TDI. The median method may utilize the median value of carbohydrate-compensation insulin dosages obtained from the historical data or the history of average TDI values.

Additionally, or optionally, at 220, the process 200 may check the degree of correlation between estimated carbohydrate insulin dosage and TDI. The AP application may use the degree of correlation between the estimated carbohydrate insulin dosage and the TDI in the building of the regression models utilizing either the kernel density estimation from 222 or the median of the IC ratios that correspond to each TDI value in 224. Alternatively, other statistical methods can be utilized, such as mean.

In the example, the data obtained at 220 is used to determine an average bolus using either the kernel density estimation 222 or the median 224.

Using the output from either the kernel density estimation 222 or the median 224, the process 200 may initialize a carbohydrate-compensation insulin dosage predictive model, which may be a linear regression model, related to TDI to predict carbohydrate-compensation insulin dosage for new users. The linear regression model may later be updated based on the user's post meal performance (e.g., the user's body's ability to return its blood glucose to within a range of a target blood glucose setting for the user). For example, at 230, the AP application my estimate the carbohydrate-compensation Insulin using an equation such as: a*TDI+b, where a is percentage of the total daily insulin (TDI) and b is the interception of this linear function, which is the adjustment for TDI related bolus prediction. It may be a negative half unit or a negative 1 unit, for example.

Metrics may be, for example, the percentage of hyperglycemic/hypoglycemic events, time in range of target blood glucose setting (e.g., within 10-20% of target blood glucose setting), average blood glucose measurement value, or the like. The AP application may be configured based on the received metrics to decide a percentage (%) of carbohydrate-compensation insulin dosage to deliver to avoid high occurrence of hypoglycemia. A high occurrence of hypoglycemia may be subjective based on the user, but an example setting be approximately 50% or the like. Alternatively, the high occurrence may be a range of percentage (%), such as 50% to 100%, in 10% increments or the like. The metrics may further include a percentage (%) of both hyperglycemic and hypoglycemic events and a determination of the marginal benefit of reducing delivery percentage (%), where a marginal benefit may be, for example, how much hypoglycemia or hyperglycemia is reduced based on the percentage of the carbohydrate-compensation insulin dosage.

At 230 in process 200, each user's typical dosage of carbohydrate-compensation insulin calculated in 222 and average TDI from 220 may be used as inputs to train a carbohydrate-compensation insulin dosage predictive model as indicated at 240. For example, the output from the carbohydrate-compensation insulin dosage predictive model at 240 may be a dosage of carbohydrate-compensation insulin calculated based on a relationship between a median carbohydrate-compensation insulin dosage and TDI.

After assessing the estimated carbohydrate-compensation dosage, to increase safety of the estimated carbohydrate-compensation dosage, the AP application may further reduce, at 250, the estimated carbohydrate-compensation dosage based on an output from the carbohydrate-compensation insulin dosage predictive model (e.g., (a*TDI+b)). The outputted percentage may be used to determine a revised carbohydrate-compensation insulin dosage. For example, an equation for such a calculation may be:

(a*TDI+b)*X_reduction+X_baseline,

where X_reduction represents the extent % reduction of the carbohydrate-compensation insulin dosage that may increase based on the output of the carbohydrate-compensation insulin dosage, and X_reduction is the same reduction that is applied at all TDI values. For example, X_baseline may be 50%, whereas X_reduction may be 0.5, where the amount of reduction is increased by 0.5% for each unit of insulin that is recommended, given the increased likelihood of potential overdelivery for larger bolus amounts. In one or more examples, the revised carbohydrate-compensation insulin dosage may be an updated carbohydrate-compensation insulin dosage.

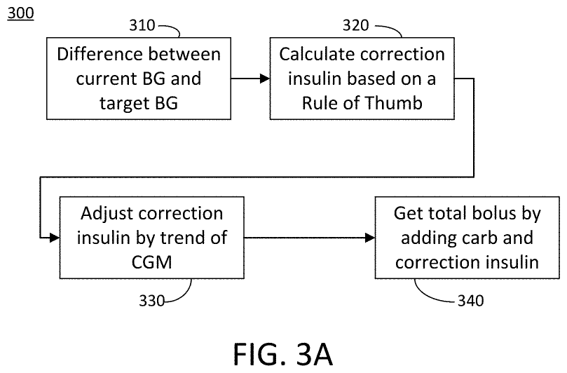

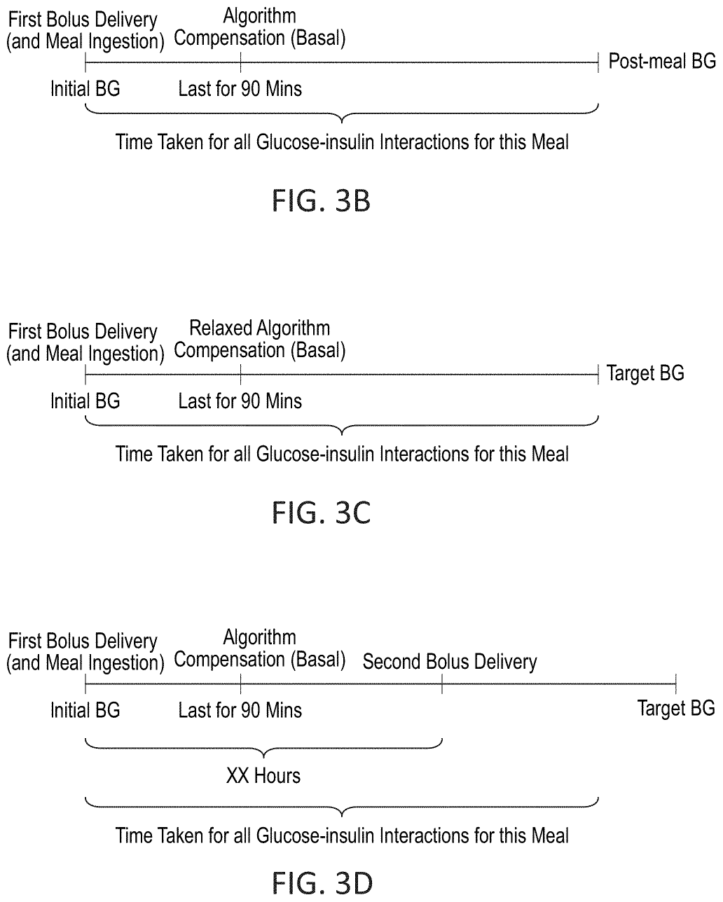

In a further example, the determination of the total bolus to be delivered may be determined differently dependent upon a type of insulin being used by a user as shown in FIG. 3A. There are known rules of thumb that may be used to assist a user in calculating an expected drop in blood glucose per unit of insulin the user receives. For regular insulin, a rule of thumb may be the 1500 rule, which is a way of calculating a user's insulin sensitivity. The 1500 rule for a user of regular (or long-acting) insulin gives an approximation of how much the user's blood sugar is expected to drop for each unit of regular insulin. In an example, the number 1500 is divided by the user's daily dosage of insulin and the quotient is used in a ratio of insulin to blood glucose. For example, if a user takes 30 units of regular insulin daily, the result of 1500 divided by 30 may represent the expected drop in blood glucose per unit of regular (or long-acting) insulin the user receives. The quotient of this division operation equals 50. Thus, in this specific example, the quotient 50 means the user's insulin sensitivity factor is 1:50, or that one unit of regular insulin will lower the respective user's blood sugar by about 50 mg/dL.

Alternatively, the rule of thumb may be different for short-acting insulin. For example, a rule of thumb may be the 1800 rule that may be used to approximate a user's insulin sensitivity to short-acting insulin. The 1800 rule for a user of short-acting insulin gives an approximation how much the user's blood sugar is expected to drop for each unit of short-acting insulin. For example, if a user takes 30 units of regular insulin daily, the result of 1800 divided by 30 may represent the expected drop in blood glucose per unit of short-acting insulin the user receives. The quotient of this division operation equals 60. Thus, in this specific example, the quotient 60 means the user's insulin sensitivity factor is 1:60, or that one unit of short-acting insulin will lower the respective user's blood sugar by about 60 mg/dL. Either the 1500 rule or the 1800 rule may be used to estimate a correction insulin dosage that may be sufficient to cover a gap between starting blood glucose value and a target blood glucose setting.

FIG. 3A illustrates a process to estimate a correction insulin dosage that accounts for an amount of insulin on-board for the user. In the process 300, an AP application may estimate the correction insulin dosage based on the 1800 rule, a trend of the blood glucose measurement values from a blood glucose monitor and a pre-existing JOB. Recall the connection insulin dosage may be used as an adjustment for meal insulin correction bolus.

In order to estimate a dose of correction insulin needed to cover the gap of starting and target BG, a difference between a current blood glucose measurement value and a target blood glucose setting may be determined by the AP application, as indicated at 310. For example, a current blood glucose measurement value (i.e., BGCurrent) may be obtained from a blood glucose monitor or from a memory that stores the most recently received blood glucose measurement value. In addition, the target blood glucose setting (i.e., BGTarget) of the user may be retrieved from a memory as well.

The AP application may make the calculation of the difference between a current blood glucose measurement value and the target blood glucose setting. At 320, the AP application, in response to the difference between the current blood glucose measurement value and the target blood glucose setting, may calculate a preliminary correction insulin dosage. “Preliminary” may refer to a correction insulin dosage that has not yet been delivered. Depending on the type of insulin (i.e., short-acting insulin or regular/long-acting insulin) the user is using, the user's insulin sensitivity may be determined using either the 1800 rule for short-acting insulin or the 1500 rule for regular insulin. The user's insulin sensitivity is determined as explained above as a Rule of Thumb. The correction insulin dosage at 320 may be estimated using the logic (in this example, using the 1800 rule) as:

$$Estimated Correction Insulin = \frac{{BG}_{Current} - {BG}_{target}}{\frac{1800}{TDI}} - IOB ,$$

where IOB may be a calculation of an amount of insulin that has not effectively been utilized by the body, TDI is total daily insulin, and the 1800 is from the 1800 rule. The 1800 is a more conservative estimate than the 1500 Rule, which may be used for regular insulin. In the example, the IOB may account for insulin in the user's body regardless of whether the insulin was provided by a basal dosage, a correction bolus, or a carbohydrate-compensation bolus. As such, IOB accounts for all preexisting insulin in the user's blood.

It is noted that the correction bolus may be used to eliminate the difference of a current blood glucose measurement value and the target blood glucose setting, and a meal bolus or carbohydrate-compensation bolus may be used to control the increase in blood glucose caused by the intake of carbohydrates. When a bolus is delivered in response to a user having a meal, the amount of insulin in the total bolus dosage delivered is equal to the carbohydrate-compensation bolus dosage plus (+) the correction bolus dosage minus (−) insulin on board (JOB).

The AP application executed by a processor may be operable to adjust the correction insulin dose to avoid hypoglycemia and hyperglycemia using the trend of the user' blood glucose measurement values provided by a blood glucose monitor. At 330, the AP application may adjust the preliminary correction insulin dosage based on a trend of blood glucose measurement values received over a predetermined period of time. In an example, the predetermined period of time is measured over a course of minutes. A blood glucose sensor, such as a CGM, may provide a trend indication. The AP application, in some instances, may interpret the trend indication and may cause the presentation of a trend indicator icon on a graphical user interface. The trend indicator icon may, for example, be an up arrow (i.e., vertical arrow pointing upwards), down arrow (i.e., vertical arrow pointing downwards), a dash (indicating no change or a flat trend), an arrow at a 45 degree upward angle or 45 degree downward angle, or the like. The angle of the upward or downward arrow may correspond to a slope or a degree of change in determined or estimated blood glucose values. For example, a 60 degree upward arrow may indicate a more rapid change in blood glucose than a 30 degree upward arrow displayed on a graphical user interface. In 330, for example, if the trend of blood glucose is downward, the correction insulin dose may be reduced by X % (which may be applied as a decimal). Alternatively, if the trend of blood glucose is upward, the correction insulin dose may be increased by Y %, where X and Y may be different and between 10%-70%, for example. The execution of the equation may output the adjusted preliminary correction insulin dosage as the estimated correction insulin dosage.

An example equation for adjusted correction insulin dosage may be:

$$( \frac{{BG}_{starting} - {BG}_{target}}{\frac{1800}{TDI}} - IOB ) * ( X or Y ) \%$$

After obtaining an adjusted correction insulin dosage, the AP application may determine a total bolus dosage. For example, the AP application may be operable to combine adjusted correction insulin with reduced carbohydrate-compensation insulin dosage, which may be as adding the adjusted correction insulin dosage to the carbohydrate-compensation insulin dosage.

Different options may be provided to enable the AP application to determine an amount of basal insulin to be delivered to bring the blood glucose measurement value into a preset blood glucose measurement range. For example, an algorithm within the AP application may adjust basal insulin for a period of time to actively compensate for under and over bolusing by determining whether a user's post-meal blood glucose measurement value is above or below a user's target blood glucose setting. An assumption may be that any initial bolus delivery that is over- or under-the optimal value is compensated by up to a maximum compensated amount possible.

In the FIG. 3B example, the user's post-meal blood glucose measurement value may be evaluated with respect to the user's target blood glucose setting. For example, the AP application may note the time of a meal notification and may obtain a post-meal blood glucose measurement value (and a blood glucose trend indication) from a blood glucose sensor. The AP application may retrieve the user's target blood glucose setting from a memory coupled to the processor that is executing the AP application. The AP application may compare the received blood glucose measurement value to a retrieved target blood glucose setting for the user. In addition, the AP application may determine whether the blood glucose trend indication is upward or downward.

In the example of FIG. 3B, the logic may proceed as follows: if the post-meal blood glucose measurement value is less than (<) target blood glucose setting, the AP application may suspend basal insulin for the peak time of insulin delivery, which may be approximately 1.5 hours, until basal insulin is higher than Δ_BG+up to a preset minimum threshold measured in mg/dL). The preset minimum compensation threshold may be, for example, 50 mg/dL, 55.0 mg/dL, 67.25 mg/dL, or the like. In addition, or alternatively, the present minimum threshold may be modifiable based on the user's insulin sensitivity or the like.

If post-meal BG is greater than (>) target BG, the AP application may be configured to deliver, for example, approximately 4 times the amount of basal insulin scheduled to be delivered over a peak time of insulin delivery with regard to the meal over some time, e.g., over 1.5 hours, until the BG reaches a threshold such as (Δ_BG−up to preset maximum compensation threshold measured in mg/dL). For example, the AP application may begin delivering this increased basal dosage of insulin for a set period of time (e.g., the peak time for insulin delivery). The delivery of the basal dosage may begin after the delivery of the sum of the estimated carbohydrate-compensation dosage of insulin and the correction insulin dosage.

As an alternative, the AP application may utilize blood glucose trend indication to determine whether the total bolus under-delivered insulin. For example, the AP application may receive a blood glucose trend indication from a blood glucose sensor. The AP application may evaluate the blood glucose trend indication with respect to a user's target blood glucose setting. A result of the evaluation of the user's blood glucose trend indication may indicate a user's blood glucose measurements are trending upward toward or over the user's target blood glucose setting. Based on the result of the evaluation, the AP application may determine the total bolus underdelivered insulin and may generate an indication that the total bolus under-delivered insulin. Conversely, the AP application may evaluate the user's blood glucose measurement value with respect to the user's target blood glucose setting. Based on a result of the evaluation indicating the user's blood glucose measurement value is less than the user's target blood glucose setting insulin, the AP application may determine the total bolus did not under-deliver insulin and may generate an indication that the total bolus did not under-deliver insulin.

Alternatively, FIG. 3C illustrates another example of logic when delivering basal insulin to bring blood glucose measurement value into set blood glucose measurement range. In the example, the AP application may be operable to cause the wearable automatic drug delivery device to begin delivery of a basal dosage of insulin that may be modified based on a relaxed safety constraint after delivering the sum of the estimated carbohydrate-compensation dosage of insulin and the correction insulin dosage. For example, in FIG. 3C, if the post-meal blood glucose measurement value is greater than (>) the target blood glucose setting, the AP application may cause a relaxing of algorithm constraints of basal insulin compensation from, for example, 4 times to 4+n times. The “n” may be a value that is determined based on a user's blood glucose settings. The ‘n’ may be determined by the following formula:

$$n = \frac{post meal BG - target BG}{N * insulin sensitivity * basal / 5 min} - 4$$

where N is the number of insulin deliveries with compensation. For example, if the compensation lasts for 1 hour, N=12, representing 12 5-minute intervals.

In yet another example of the determination of a basal insulin dosage for delivery to bring the user's blood glucose measurement value into a set blood glucose measurement range is shown in the example of FIG. 3D. The example process of FIG. 3D may be implemented if the post-meal BG is higher than target BG. In an instance where the post-meal BG some period of time after meal ingestion is higher than target BG, the AP application may be operable to cause a second bolus to be delivered XX hours after the delivery of an initial bolus to compensate for carbohydrates at time of meal ingestion. In the example, the AP application may cause delivery of a second, or secondary, bolus a set period of time (shown as XX) after delivering the sum of the estimated carbohydrate-compensation dosage of insulin and the correction insulin dosage. In the example, the set period of time XX may, for example, be equal to approximately 2 hours, 3 hours, 4 hours, or greater.

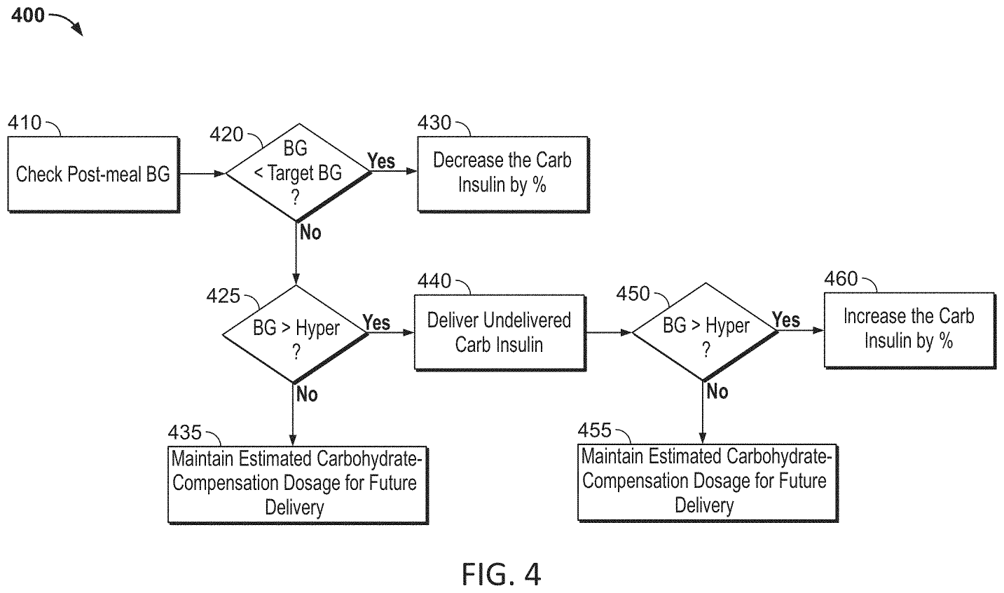

The AP application may perform additional functions. In some instances, the carbohydrate-compensation insulin dosage may be adapted as mentioned in the examples of FIGS. 1A and 1B. The details of the adaptation of the carbohydrate-compensation dosage of insulin by a predetermined factor may be described with reference to FIG. 4.

The process 400 example of FIG. 4 may be considered a long-term update of the carbohydrate-compensation insulin dosage based on past hypoglycemia and hyperglycemia events for a future bolus. The update may be applied to a next bolus that may include a carbohydrate-compensation insulin dosage.

In an operational example, when a carbohydrate-compensation insulin dosage, which may be considered a bolus, is to be delivered. The AP application may deliver only a partial dosage of the carbohydrate-compensation insulin dosage. For example, the AP application may cause a percentage, such as 60-80 percent, of the estimated carbohydrate-compensation insulin dosage to be held in reserve as a reserve dosage. The partial dosage and the reserve dosage when summed together from an entire amount of insulin in the estimated carbohydrate-compensation insulin dosage. The estimation of the estimated carbohydrate-compensation insulin dosage may be confirmed and evaluated according to the process 400. For example, delivery of the reduced or partial dosage of the estimated carbohydrate-compensation dosage of insulin permits the AP application to determine if the user's body's reaction to the insulin may still compensate for the carbohydrates from the ingested meal. In addition, the delivery of only a portion provides the benefit of ensuring the AP application does not over deliver insulin to the user.

In process 400, the AP application may monitor the user's post-meal blood glucose by obtaining a blood glucose measurement value from a blood glucose sensor, as indicated at 410. At 410, the AP application may be configured to receive blood glucose measurement values from a continuous blood glucose monitor approximately every five minutes or the like. In further examples, the AP application may also receive a blood glucose trend indication and other information. At 420, the AP application may determine whether the blood glucose measurement value is below a target blood glucose setting for the user. An example of a target blood glucose setting may be approximately 120 mg/dL, which may have an upper boundary, such as 140 mg/dL, and a lower boundary, such as 100 mg/dL. Based on the response at 420, the AP application may take different actions. For example; if post-meal blood glucose measurement value is less than (<) the target blood glucose setting, the process may proceed to 430. At 430, the AP application may update the estimated carbohydrate-compensation insulin dosage by decreasing the estimated carbohydrate-compensation insulin dosage by a preset percentage value, such as 1-10%, for a next delivery of a carbohydrate-compensation insulin dosage (i.e., when a next meal is ingested by the user).

Alternatively, at 420, the determination is that the post-meal blood glucose measurement value is greater than (>) the target blood glucose setting. In response to this determination, the process 400 may proceed from 420 to 425.

At 425, the AP application may determine whether the post-meal blood glucose measurement value falls in the range of the target blood glucose setting by determining whether the post-meal blood glucose measurement value is less than (i.e., below or at) a predetermined blood glucose hyperglycemia threshold. For example, the AP application may determine whether the post-meal blood glucose measurement value is below 180 mg/dL, which may be the predetermined blood glucose hyperglycemia threshold (also referred to as the “hyperglycemia threshold” or “HYPER”). If the post-meal blood glucose measurement value is below the hyperglycemia threshold HYPER (e.g., 180 mg/dL, a user's specific hyperglycemia threshold, or the like), the AP application, at 435, may keep the estimated carbohydrate-compensation insulin dosage for causing future delivery of a meal compensation bolus dosage corresponding to the estimated carbohydrate-compensation insulin dosage.

However, if, at 425, the AP application determines the post-meal blood glucose measurement value is greater than (>) the hyperglycemia threshold HYPER despite the delivery of the percentage of the estimated carbohydrate-compensation insulin dosage, the AP application may proceed to 440. Since only a percentage of the estimated carbohydrate-compensation insulin dosage was delivered as a bolus in response to the meal notification or announcement, additional insulin remains to be delivered. At 440, the AP application may cause delivery of the remaining percentage (e.g., the remaining 40-20%) of estimated meal bolus.

The process 400 after 440 may proceed to 450. At 450, the AP application may determine whether the delivery of the remaining percentage of the estimated carbohydrate-compensation insulin dosage reduced the user's blood glucose. The AP application may, after delivery of the remaining percentage of the estimated carbohydrate-compensation, wait a period of time (e.g., 90-120 minutes) to permit the remaining percentage of the estimated carbohydrate-compensation insulin dosage to have an effect on the user's blood glucose. After the passage of the period of time, the AP application may use a subsequently-received blood glucose measurement value from a blood glucose monitor sometime to determine whether the subsequent blood glucose measurement value (which is a post-meal blood glucose measurement value) is less than the upper boundary of the target blood glucose measurement.

At 450, the AP application may compare the estimated blood glucose value to a predetermined blood glucose hyperglycemia threshold HYPER. Based on a determination from the comparison that the post-meal blood glucose measurement value is less than (<) the hyperglycemia threshold the process may proceed to 455. At 455, the AP application may maintain the estimated carbohydrate-compensation insulin dosage for causing future delivery of a meal compensation bolus dosage corresponding to the estimated carbohydrate-compensation insulin dosage.

Alternatively, if, at 450, the blood glucose measurement value is still greater than (>) the upper boundary of the target blood glucose setting, the AP application may proceed to 460. At 460, the AP application be operable to update the estimated meal bolus by increasing the percentage of insulin in the carbohydrate-compensation insulin dosage for next delivery.

For example, at 460, the AP application may increase the estimated carbohydrate-compensation dosage by a predetermined percentage of the estimated carbohydrate-compensation dosage in response to the estimated blood glucose value being greater than the predetermined blood glucose hyperglycemia threshold. In an example, the increased percentage may be 5%-10% for each condition iteration.

It may be helpful to discuss an example of a drug delivery system that may implement the techniques described with reference to the examples of FIGS. 1A-4.

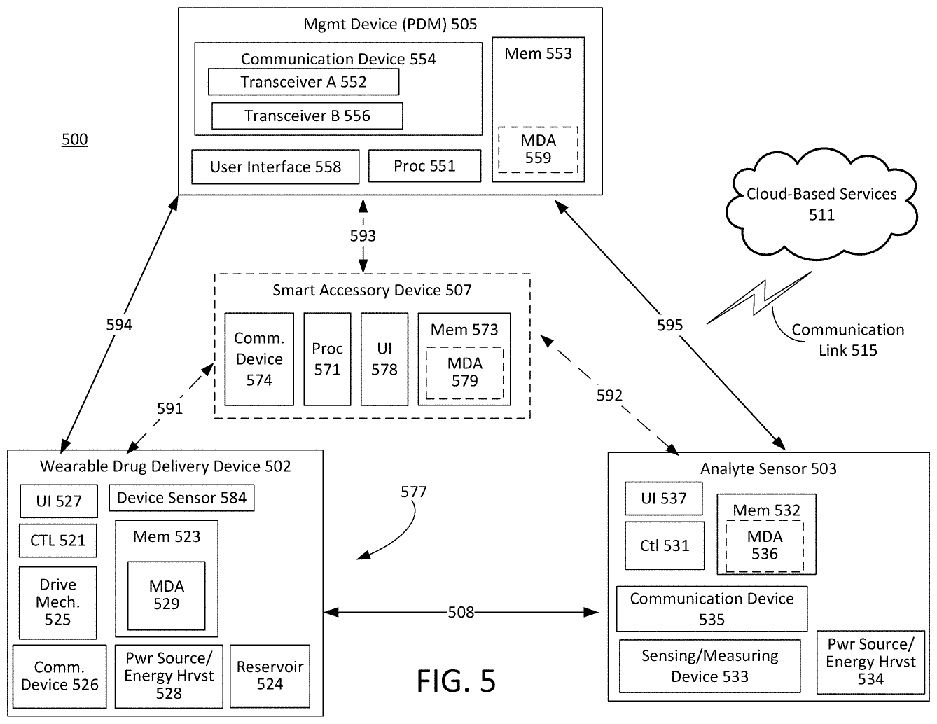

FIG. 5 illustrates a functional block diagram of a system example suitable for implementing the example processes and techniques described herein.

The automatic drug delivery system 500 may implement (and/or provide functionality for) a medication delivery algorithm, such as an artificial pancreas (AP) application, to govern or control automated delivery of a drug or medication, such as insulin, to a user (e.g., to maintain euglycemia—a normal level of glucose in the blood). The drug delivery system 500 may be an automated drug delivery system that may include a wearable automatic drug delivery device 502, an analyte sensor 503, and a management device (PDM) 505.

The system 500, in an optional example, may also include a smart accessory device 507, such as a smartwatch, a personal assistant device or the like, which may communicate with the other components of system 500 via either a wired or wireless communication links 591-593.

The management device 505 may be a computing device such as a smart phone, a tablet, a personal diabetes management device, a dedicated diabetes therapy management device, or the like. In an example, the management device (PDM) 505 may include a processor 551, a management device memory 553, a user interface 558, and a communication device 554. The management device 505 may contain analog and/or digital circuitry that may be implemented as a processor 551 for executing processes based on programming code stored in the management device memory 553, such as the medication delivery algorithm or application (MDA) 559, to manage a user's blood glucose levels and for controlling the delivery of the drug, medication, or therapeutic agent to the user as well as other functions, such as calculating carbohydrate-compensation dosage, a correction bolus dosage and the like as discussed above. The management device 505 may be used to program, adjust settings, and/or control operation of the wearable automatic drug delivery device 502 and/or the analyte sensor 503 as well as the optional smart accessory device 507.

The processor 551 may also be configured to execute programming code stored in the management device memory 553, such as the MDA 559. The MDA 559 may be a computer application that is operable to deliver a drug based on information received from the analyte sensor 503, the cloud-based services 511 and/or the management device 505 or optional smart accessory device 507. The memory 553 may also store programming code to, for example, operate the user interface 558 (e.g., a touchscreen device, a camera or the like), the communication device 554 and the like. The processor 551 when executing the MDA 559 may be configured to implement indications and notifications related to meal ingestion, blood glucose measurements, and the like. The user interface 558 may be under the control of the processor 551 and be configured to present a graphical user interface that enables the input of a meal announcement, adjust setting selections and the like as described above.

In a specific example, when the MDA 559 is an artificial pancreas (AP) application, the processor 551 is also configured to execute a diabetes treatment plan (which may be stored in a memory) that is managed by the MDA 559 stored in memory 553. In addition to the functions mentioned above, when the MDA 559 is an AP application, it may further provide functionality to enable the processor 551 to determine a carbohydrate-compensation dosage, a correction bolus dosage and determine a basal dosage according to a diabetes treatment plan. In addition, as an AP application, the MDA 559 provides functionality to enable the processor 551 to output signals to the wearable automatic drug delivery device 502 to deliver the determined bolus and basal dosages described with reference to the examples of FIGS. 1A-4.

The communication device 554 may include one or more transceivers such as Transceiver A 552 and Transceiver B 556 and receivers or transmitters that operate according to one or more radio-frequency protocols. In the example, the transceivers 552 and 556 may be a cellular transceiver and a Bluetooth® transceiver, respectively. For example, the communication device 554 may include a transceiver 552 or 556 configured to receive and transmit signals containing information usable by the MDA 559.

The wearable automatic drug delivery device 502, in the example system 500, may include a user interface 527, a controller 521, a drive mechanism 525, a communication device 526, a memory 523, a power source/energy harvesting circuit 528, device sensors 584, and a reservoir 524. The wearable automatic drug delivery device 502 may be configured to perform and execute the processes described in the examples of FIGS. 1A-4 without input from the management device 505 or the optional smart accessory device 507. As explained in more detail, the controller 521 may be operable, for example, implement the processes of FIGS. 1A-4 as well as determine an amount of insulin delivered, JOB, insulin remaining, and the like. The controller 521 alone may implement the processes of FIGS. 1A-4 as well as determine an amount of insulin delivered, JOB, insulin remaining, and the like, such as control insulin delivery, based on an input from the analyte sensor 504.

The memory 523 may store programming code executable by the controller 521. The programming code, for example, may enable the controller 521 to control expelling insulin from the reservoir 524 and control the administering of doses of medication based on signals from the MDA 529 or, external devices, if the MDA 529 is configured to implement the external control signals.

The reservoir 524 may be configured to store drugs, medications or therapeutic agents suitable for automated delivery, such as insulin, morphine, blood pressure medicines, chemotherapy drugs, or the like.

The device sensors 584 may include one or more of a pressure sensor, a power sensor, or the like that are communicatively coupled to the controller 521 and provide various signals. For example, a pressure sensor of the device sensors 584 may be configured to provide an indication of the fluid pressure detected in a fluid pathway between a needle or cannula (shown in examples of FIGS. 2A and 2B)) inserted in a user and the reservoir 524. For example, the pressure sensor may be coupled to or integral with a needle/cannula insertion component (which may be part of the drive mechanism 525) or the like. In an example, the controller 521 or a processor, such as 551, may be operable to determine that a rate of drug infusion based on the indication of the fluid pressure. The rate of drug infusion may be compared to an infusion rate threshold, and the comparison result may be usable in determining an amount of insulin onboard (JOB) or a total daily insulin (TDI) amount.

In an example, the wearable automatic drug delivery device 502 includes a communication device 526, which may be a receiver, a transmitter, or a transceiver that operates according to one or more radio-frequency protocols, such as Bluetooth, Wi-Fi, a near-field communication standard, a cellular standard, or the like. The controller 521 may, for example, communicate with a personal diabetes management device 505 and an analyte sensor 503 via the communication device 526.

The wearable automatic drug delivery device 502 may be attached to the body of a user, such as a patient or diabetic, at an attachment location and may deliver any therapeutic agent, including any drug or medicine, such as insulin or the like, to a user at or around the attachment location. A surface of the wearable automatic drug delivery device 502 may include an adhesive to facilitate attachment to the skin of a user as described in earlier examples.

The wearable automatic drug delivery device 502 may, for example, include a reservoir 524 for storing the drug (such as insulin), a needle or cannula (not shown in this example) for delivering the drug into the body of the user (which may be done subcutaneously, intraperitoneally, or intravenously), and a drive mechanism 525 for transferring the drug from the reservoir 524 through a needle or cannula and into the user. The drive mechanism 525 may be fluidly coupled to reservoir 524, and communicatively coupled to the controller 521.

The wearable automatic drug delivery device 502 may further include a power source 528, such as a battery, a piezoelectric device, other forms of energy harvesting devices, or the like, for supplying electrical power to the drive mechanism 525 and/or other components (such as the controller 521, memory 523, and the communication device 526) of the wearable automatic drug delivery device 502.

In some examples, the wearable automatic drug delivery device 502 and/or the management device 505 may include a user interface 558, respectively, such as a keypad, a touchscreen display, levers, light-emitting diodes, buttons on a housing of the management device 505, a microphone, a camera, a speaker, a display, or the like, that is configured to allow a user to enter information and allow the management device 505 to output information for presentation to the user (e.g., alarm signals or the like). The user interface 558 may provide inputs, such as a voice input, a gesture (e.g., hand or facial) input to a camera, swipes to a touchscreen, or the like, to processor 551 which the programming code interprets.

When configured to communicate to an external device, such as the PDM 505 or the analyte sensor 504, the wearable automatic drug delivery device 502 may receive signals over the wired or wireless link 594 from the management device (PDM) 505 or 508 from the analyte sensor 504. The controller 521 of the wearable automatic drug delivery device 502 may receive and process the signals from the respective external devices as described with reference to the examples of FIGS. 1A-4 as well as implementing delivery of a drug to the user according to a diabetes treatment plan or other drug delivery regimen.

In an operational example, the processor 521 when executing the MDA 559 may output a control signal operable to actuate the drive mechanism 525 to deliver a carbohydrate-compensation dosage of insulin, a correction bolus, a revised basal dosage or the like as described with reference to the examples of FIGS. 1A-4.

The smart accessory device 507 may be, for example, an Apple Watch®, other wearable smart device, including eyeglasses, provided by other manufacturers, a global positioning system-enabled wearable, a wearable fitness device, smart clothing, or the like. Similar to the management device 505, the smart accessory device 507 may also be configured to perform various functions including controlling the wearable automatic drug delivery device 502. For example, the smart accessory device 507 may include a communication device 574, a processor 571, a user interface 578 and a memory 573. The user interface 578 may be a graphical user interface presented on a touchscreen display of the smart accessory device 507. The memory 573 may store programming code to operate different functions of the smart accessory device 507 as well as an instance of the MDA 579. The processor 571 that may execute programming code, such as site MDA 579 for controlling the wearable automatic drug delivery device 502 to implement the FIG. 1A-4 examples described herein.

The analyte sensor 503 may include a controller 531, a memory 532, a sensing/measuring device 533, a user interface 537, a power source/energy harvesting circuitry 534, and a communication device 535. The analyte sensor 503 may be communicatively coupled to the processor 551 of the management device 505 or controller 521 of the wearable automatic drug delivery device 502. The memory 532 may be configured to store information and programming code, such as an instance of the MDA 536.

The analyte sensor 503 may be configured to detect multiple different analytes, such as lactate, ketones, uric acid, sodium, potassium, alcohol levels or the like, and output results of the detections, such as measurement values or the like. The analyte sensor 503 may, in an example, be configured to measure a blood glucose value at a predetermined time interval, such as every 5 minutes, or the like. The communication device 535 of analyte sensor 503 may have circuitry that operates as a transceiver for communicating the measured blood glucose values to the management device 505 over a wireless link 595 or with wearable automatic drug delivery device 502 over the wireless communication link 508. While called an analyte sensor 503, the sensing/measuring device 533 of the analyte sensor 503 may include one or more additional sensing elements, such as a glucose measurement element a heart rate monitor, a pressure sensor, or the like. The controller 531 may include discrete, specialized logic and/or components, an application-specific integrated circuit, a microcontroller or processor that executes software instructions, firmware, programming instructions stored in memory (such as memory 532), or any combination thereof.

Similar to the controller 521, the controller 531 of the analyte sensor 503 may be operable to perform many functions. For example, the controller 531 may be configured by the programming code stored in the memory 532 to manage the collection and analysis of data detected the sensing and measuring device 533.

Although the analyte sensor 503 is depicted in FIG. 5 as separate from the wearable automatic drug delivery device 502, in various examples, the analyte sensor 503 and wearable automatic drug delivery device 502 may be incorporated into the same unit. That is, in various examples, the sensor 503 may be a part of the wearable automatic drug delivery device 502 and contained within the same housing of the wearable automatic drug delivery device 502 (e.g., the sensor 503 or, only the sensing/measuring device 533 and memory storing related programming code may be positioned within or integrated into, or into one or more components, such as the memory 523, of, the wearable automatic drug delivery device 502). In such an example configuration, the controller 521 may be able to implement the process examples of FIGS. 1A-4 alone without any external inputs from the management device 505, the cloud-based services 511, another sensor (not shown), the optional smart accessory device 507, or the like.

The communication link 515 that couples the cloud-based services 511 to the respective devices 502, 503, 505 or 507 of system 500 may be a cellular link, a Wi-Fi link, a Bluetooth link, or a combination thereof. Services provided by cloud-based services 511 may include data storage that stores anonymized data, such as blood glucose measurement values, historical IOB or TDI, prior carbohydrate-compensation dosage, and other forms of data. In addition, the cloud-based services 511 may process the anonymized data from multiple users to provide generalized information related to TDI, insulin sensitivity, IOB and the like.

The wireless communication links 508, 591, 592, 593, 594 and 595 may be any type of wireless link operating using known wireless communication standards or proprietary standards. As an example, the wireless communication links 508, 591, 592, 593, 594 and 595 may provide communication links based on Bluetooth®, Zigbee®, Wi-Fi, a near-field communication standard, a cellular standard, or any other wireless protocol via the respective communication devices 554, 574, 526 and 535.

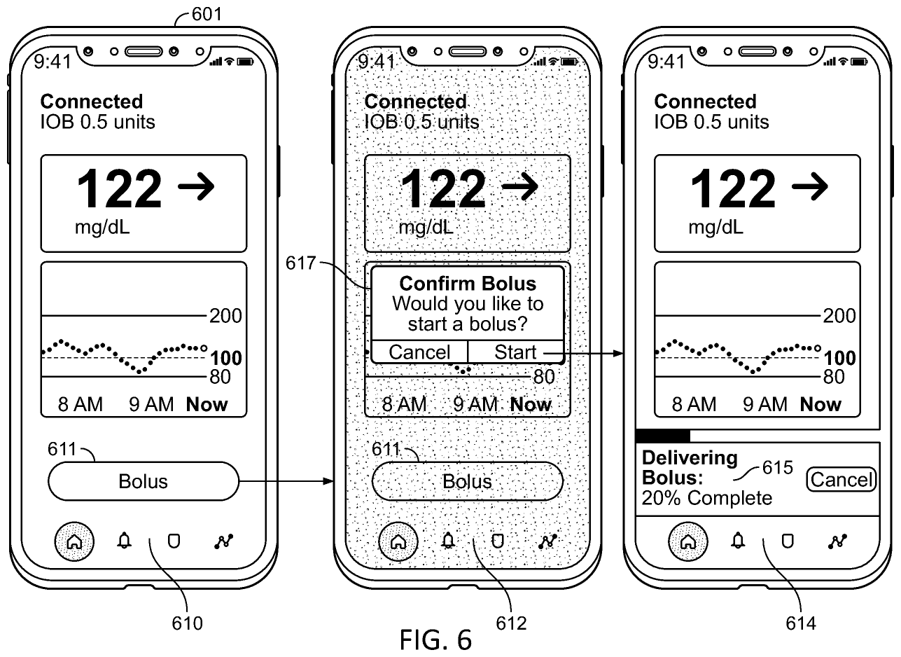

FIG. 6 illustrates an example of a graphical user interface usable with the disclosed techniques and devices.

A management device as described in earlier examples may be implemented as management device 601, which may be a dedicated computing device having a form factor similar to a smart phone or may be a smart phone that is operable to execute a mobile computer application that implements some or all of the meal announcement features described herein. The management device 601 may be operable to implement a graphical user interface, such as 610. The graphical user interface may include user activated inputs, such as bolus button 611 as well as other inputs.

In an example, an MDA application as described with reference to an earlier example may be operable to receive a meal announcement. For example, the meal announcement may be a notification of ingestion of a meal provided by the user via a user input or an automated meal detection algorithm. In the example of FIG. 6, the meal announcement may be in response to a user engaging with the bolus button 611. In response to the user interaction with bolus button 611, the algorithms of the MDA application may cause generation of a confirmation user interface 612 that is an update to the graphical user interface 610. The confirmation user interface 612 may include a confirmation button 617 to be presented to allow the user to confirm ingestion of the meal. In response to the confirmation of the meal, the confirmation user interface 612 may be modified to present a meal announcement response graphical user interface 614. The meal announcement response graphical user interface 614 may include an indicator 615 of a bolus dosage that may be delivered in response to the meal announcement.

While the button 611 and confirmation button 617 in the example of FIG. 6 utilizes the word “bolus,” the phrasing on such buttons may be different. For example, button 611 may state “Announce Meal,” or “Meal Announcement,” or may ask a question, such as “Are you having a meal?” or “Announce Meal?” And button 617 may similarly state corresponding language to confirm the meal announcement or bolus request, such as “Confirm meal announcement” adjacent explanatory text, such as “Would you like to start a bolus?” or the like. The default size of the bolus (e.g., in number of units) may also be depicted in the confirmation screen or confirmation button 617. Moreover, the default size of the bolus may be configured in a settings portion of the application.

Software related implementations of the techniques described herein, such as the processes examples described with reference to FIGS. 1A-4 may include, but are not limited to, firmware, application specific software, or any other type of computer readable instructions that may be executed by one or more processors. The computer readable instructions may be provided via non-transitory computer-readable media. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

In addition, or alternatively, while the examples may have been described with reference to a closed loop algorithmic implementation, variations of the disclosed examples may be implemented to enable open loop use. The open loop implementations allow for use of different modalities of delivery of insulin such as smart pen, syringe or the like. For example, the disclosed AP application and algorithms may be operable to perform various functions related to open loop operations, such as the generation of prompts requesting the input of information such as weight or age. Similarly, a dosage amount of insulin may be received by the AP application or algorithm from a user via a user interface. Other open-loop actions may also be implemented by adjusting user settings or the like in an AP application or algorithm.

Some examples of the disclosed device or processes may be implemented, for example, using a storage medium, a computer-readable medium, or an article of manufacture which may store an instruction or a set of instructions that, if executed by a machine (i.e., processor or controller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

Program aspects of the technology may be thought of as “products” or “articles of manufacture” typically in the form of executable code and/or associated data that is carried on or embodied in a type of non-transitory, machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects. The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A method, comprising:
receiving a meal announcement, wherein the meal announcement is notification of ingestion of a meal;
estimating, in response to the announcement of ingestion of the meal, a carbohydrate-compensation dosage of insulin;
estimating an amount of insulin-on-board (JOB) based on insulin delivery history;
obtaining a current blood glucose measurement value;
estimating a correction insulin dosage using the estimated amount of IOB and the current blood glucose measurement value;
delivering a sum of the estimated carbohydrate-compensation dosage of insulin and the correction insulin dosage upon completion of estimating the correction insulin dosage;
monitoring changes in blood glucose measurement value over time;
delivering basal insulin to bring blood glucose measurement value into a set blood glucose measurement range;
determining, within a preset time of receiving the meal announcement, whether a blood glucose measurement value obtained within the preset time has exceeded a hyperglycemia threshold or fallen below a hypoglycemia threshold; and
in response to determining the blood glucose measurement value obtained within the preset time has exceeded a hyperglycemia threshold or fallen below a hypoglycemia threshold, adapting the carbohydrate-compensation dosage of insulin by a predetermined factor.

2. The method of claim 1, further comprising:
estimating an updated correction insulin dosage using an updated estimate of an amount of JOB; and
causing a sum of the adapted carbohydrate-compensation dosage of insulin and the updated correction insulin dosage to be delivered upon completion of the estimate of the updated correction insulin dosage.

3. The method of claim 1, wherein estimating the carbohydrate-compensation dosage of insulin further comprises:
obtaining a user's historical blood glucose measure values;
obtaining the user's estimated carbohydrate-compensation dosage and average total daily insulin;
input the user's estimated carbohydrate-compensation dosage and average total daily insulin to train the carbohydrate-compensation insulin dosage predictive model; and
reducing the estimated carbohydrate-compensation dosage based on an output from the carbohydrate-compensation insulin dosage predictive model.

4. The method of claim 3, further comprising:
determining from the user's carbohydrate-compensation dosage and average total daily insulin a typical bolus value based on a median bolus delivered in response to previous meal announcements; and
using the median bolus delivered as a factor in the calculating the difference of a total of user's carbohydrate-compensation dosage and average total daily insulin.

5. The method of claim 3, further comprising:
applying a kernel density estimation model to the user's carbohydrate-compensation dosage and average total daily insulin a typical bolus value based on a median bolus delivered in response to previous meal announcements; and
using an output of the kernel density estimation model as a factor in the calculating the difference of a total of user's carbohydrate-compensation dosage and average total daily insulin.

6. The method of claim 1, wherein estimating a correction insulin dosage further comprises:
determining a difference between a current blood glucose measurement value and a target blood glucose setting;
calculating a preliminary correction insulin dosage;
adjusting the preliminary correction insulin dosage based on a trend of blood glucose measurement values received over a predetermined period of time to provide the estimated correction insulin dosage; and
outputting the estimated correction insulin dosage for delivery to the user.

7. The method of claim 1, wherein delivering basal insulin to bring blood glucose measurement value into set blood glucose measurement range further comprises:
beginning delivery of a basal dosage of insulin after a set period of time after delivering the sum of the estimated carbohydrate-compensation dosage of insulin and the correction insulin dosage.

8. The method of claim 1, wherein delivering basal insulin to bring blood glucose measurement value into set blood glucose measurement range further comprises:
beginning delivery of a basal dosage of insulin modified based on a relaxed safety constraints after delivering the sum of the estimated carbohydrate-compensation dosage of insulin and the correction insulin dosage.

9. The method of claim 1, when delivering basal insulin to bring blood glucose measurement value into set blood glucose measurement range, further comprising:
beginning delivery of a basal dosage of insulin after delivering the sum of the estimated carbohydrate-compensation dosage of insulin and the correction insulin dosage; and
delivering a secondary bolus, a set period of time after delivering the sum of the estimated carbohydrate-compensation dosage of insulin and the correction insulin dosage.

10. The method of claim 1, wherein adapting the carbohydrate-compensation dosage of insulin by a predetermined factor further comprises:
checking post-meal blood glucose by obtaining a blood glucose measurement value from a blood glucose sensor;
determining whether the blood glucose measurement value is below a target blood glucose; and
in response to the blood glucose measurement value being below the target blood glucose, decreasing the estimated carbohydrate-compensation dosage by a preset percentage value.

11. The method of claim 1, wherein adapting the carbohydrate-compensation dosage of insulin by a predetermined factor further comprises:
delivering a partial dosage of the estimated carbohydrate-compensation dosage of insulin, wherein the partial dosage and a reserve dosage when summed together include an amount of insulin in the estimated carbohydrate-compensation dosage of insulin;
checking post-meal blood glucose by obtaining a blood glucose measurement value from a blood glucose sensor;
determining whether the blood glucose measurement value is less than a target blood glucose setting;
in response to the blood glucose measurement value being above the target blood glucose setting, determining whether the blood glucose measurement value is less than a predetermined blood glucose hyperglycemia threshold; and
in response to the blood glucose measurement value being greater than the predetermined blood glucose hyperglycemia threshold, delivering a reserve dosage of the estimated carbo carbohydrate-compensation dosage of insulin.

12. The method of the claim 11, further comprising:
after delivery of the reserve dosage the estimated carbo carbohydrate-compensation dosage of insulin, determining whether a subsequent blood glucose measurement value is less than the predetermined blood glucose hyperglycemia threshold,
in response to the blood glucose measurement value being greater than the predetermined blood glucose hyperglycemia threshold, increasing the estimated carbohydrate-compensation dosage for future delivery by a predetermined percentage of the estimated carbohydrate-compensation dosage.

13. A method, comprising:
obtaining a user's total daily insulin, a user's target blood glucose and a user's current blood glucose measurement;
estimating a carbohydrate-compensation insulin dosage using the obtained total daily insulin;
estimating a correction insulin dosage using the user's target blood glucose and the user's blood glucose measurement;
combining the carbohydrate-compensation insulin dosage and the correction insulin dosage for a total bolus;
delivering the total bolus;
monitoring status of a user's blood glucose and other information related to the user's blood glucose;
determining, based on a determination of the status of the user's blood glucose and other information related to the user's blood glucose, whether the total bolus underdelivered insulin;
determining whether to update a carbohydrate-compensation estimation algorithm based on the determination that the total bolus underdelivered insulin; and
generating, based on a determination of the status of the user's blood glucose and other information related to the user's blood glucose, an update of a future carbohydrate-compensation insulin dosage.

14. The method of the claim 13, wherein monitoring the status of the user's blood glucose and other information related to the user's blood glucose, comprises:
receiving a blood glucose measurement value and a blood glucose trend indication from a blood glucose sensor;
comparing the received blood glucose measurement value to a target blood glucose setting for the user;
determining a direction of the user's blood glucose based on whether the blood glucose trend indication indicates an upward or downward direction for the user's blood glucose; and
outputting a result of the comparing and the determination of the direction of the user's blood glucose as the status of the user's blood glucose and other information related to the user's blood glucose for use in determining whether the total bolus underdelivered insulin.

15. The method of the claim 13, wherein determining whether the total bolus underdelivered insulin, comprises:
evaluating a user's blood glucose measurement value received from a blood glucose sensor with respect to a user's target blood glucose setting;
determining the total bolus underdelivered based on a result of the evaluation indicating the user's blood glucose measurement value is greater than the user's target blood glucose setting insulin; and
generating an indication that the total bolus underdelivered insulin.

16. The method of the claim 13, wherein determining whether the total bolus underdelivered insulin comprises:
receiving a blood glucose trend indication from a blood glucose sensor;
evaluating the blood glucose trend indication with respect to a user's target blood glucose setting;
determining the total bolus underdelivered insulin based on a result of the evaluation of the blood glucose trend indication indicating a user's blood glucose measurements is trending upward toward or over a user's target blood glucose setting; and
generating an indication that the total bolus underdelivered insulin.

17. The method of the claim 13, wherein determining whether the total bolus underdelivered insulin comprises:
evaluating the user's blood glucose measurement value with respect to the user's target blood glucose setting; and
determining, based a result of the evaluation indicating the user's blood glucose measurement value is less than the user's target blood glucose setting insulin, the total bolus did not underdeliver insulin; and
generating an indication that the total bolus did not underdelivered insulin.

18. The method of the claim 13, wherein determining whether the total bolus underdelivered insulin comprises:
evaluating a trend of a user's blood glucose measurement value with respect to the user's target blood glucose setting; and
determining based on a result of the evaluation indicating the user's blood glucose measurement value is less than the user's target blood glucose setting insulin, the total bolus did not underdeliver insulin; and
generating an indication that the total bolus did not underdeliver insulin.

19. A drug delivery device, comprising:
a memory storing programming code;
a controller configured to execute the programming code, wherein the controller upon executing the programming code is configured to:
receive a meal announcement, wherein the meal announcement is notification of ingestion of a meal;
estimate, in response to the announcement of ingestion of the meal, a carbohydrate-compensation dosage of insulin;
estimate an amount of insulin-on-board (JOB) based on insulin delivery history;
obtain a current blood glucose measurement value;
estimate a correction insulin dosage using the estimated amount of IOB and the current blood glucose measurement value;
deliver a sum of the estimated carbohydrate-compensation dosage of insulin and the correction insulin dosage upon completion of estimate of correction insulin dosage;
monitor changes in blood glucose measurement value over time;
deliver basal insulin to bring blood glucose measurement value into set blood glucose measurement range;
determine, within a preset time of receiving the meal announcement, whether a blood glucose measurement value obtained within the preset time has exceeded a hyperglycemia threshold or fallen below a hypoglycemia threshold; and
in response to the blood glucose measurement value obtained within the preset time has exceeded a hyperglycemia threshold or fallen below a hypoglycemia threshold, adapting the carbohydrate-compensation dosage of insulin by a predetermined factor.

20. The drug delivery device of claim 19, wherein the controller upon executing the programming code is further configured to:
estimate an updated correction insulin dosage using an updated estimate of an amount of JOB; and
cause a sum of the adapted carbohydrate-compensation dosage of insulin and the updated correction insulin dosage to be delivered upon completion of the estimate of the updated correction insulin dosage.

21. The method of claim 19, wherein the controller upon executing the programming code, when estimating the carbohydrate-compensation dosage of insulin, is further configured to:
obtain a user's historical blood glucose measure values;
obtain the user's estimated carbohydrate-compensation dosage and average total daily insulin;
calculate difference of a total of user's carbohydrate-compensation dosage and average total daily insulin;
input the difference into carbohydrate-compensation insulin dosage predictive model; and
reduce the estimated carbohydrate-compensation dosage based on an output from the carbohydrate-compensation insulin dosage predictive model.

22. The method of claim 21, wherein the controller upon executing the programming code is further configured to:
determine from the user's carbohydrate-compensation dosage and average total daily insulin a typical bolus value based on a median bolus delivered in response to previous meal announcements; and
use the median bolus delivered as a factor in the calculating the difference of a total of user's carbohydrate-compensation dosage and average total daily insulin.

23. The drug delivery device of claim 21, wherein the controller upon executing the programming code is further configured to:
apply a kernel density estimation model to the user's carbohydrate-compensation dosage and average total daily insulin a typical bolus value based on a median bolus delivered in response to previous meal announcements; and
use an output of the kernel density estimation model as a factor in the calculating the difference of a total of user's carbohydrate-compensation dosage and average total daily insulin.

24. The drug delivery device of claim 19, wherein the controller upon executing the programming code, when estimating the carbohydrate-compensation dosage of insulin, is further configured to:
determine a difference between a current blood glucose measurement value and a target blood glucose setting;
calculate a preliminary correction insulin dosage;
adjust the preliminary correction insulin dosage based on a trend of blood glucose measurement values received over a predetermined period of time; and
output the adjusted preliminary correction insulin dosage as the estimated correction insulin dosage.

25. The drug delivery device of claim 19, when delivering basal insulin to bring blood glucose measurement value into set blood glucose measurement range, further comprising:
cause delivery of a basal dosage of insulin after a set period of time after delivering the sum of the estimated carbohydrate-compensation dosage of insulin and the correction insulin dosage.

26. The drug delivery device of claim 19, when delivering basal insulin to bring blood glucose measurement value into set blood glucose measurement range, further comprising:
cause delivery of a basal dosage of insulin modified based on a relaxed safety constraints after delivering the sum of the estimated carbohydrate-compensation dosage of insulin and the correction insulin dosage.

27. The drug delivery device of claim 19, when delivering basal insulin to bring blood glucose measurement value into set blood glucose measurement range, further comprising:
begin delivery of a basal dosage of insulin after delivering the sum of the estimated carbohydrate-compensation dosage of insulin and the correction insulin dosage; and
deliver a secondary bolus, a set period of time after delivering the sum of the estimated carbohydrate-compensation dosage of insulin and the correction insulin dosage.

28. The drug delivery device of claim 19, when adapting the carbohydrate-compensation dosage of insulin by a predetermined factor, further comprising:
check post-meal blood glucose by obtaining a blood glucose measurement value from a blood glucose sensor;
determine whether the blood glucose measurement value is below a target blood glucose; and
in response to the blood glucose measurement value being below the target blood glucose, decrease the estimated carbohydrate-compensation dosage by a preset percentage value.

29. The drug delivery device of claim 19, when adapting the carbohydrate-compensation dosage of insulin by a predetermined factor, further comprises:
deliver a partial dosage of the estimated carbohydrate-compensation dosage of insulin, wherein the partial dosage and a reserve dosage when summed together include an amount of insulin in the estimated carbohydrate-compensation dosage of insulin;
check post-meal blood glucose by obtaining a blood glucose measurement value from a blood glucose sensor;
determine whether the blood glucose measurement value is less than a target blood glucose setting;
in response to the blood glucose measurement value being above the target blood glucose setting, determine whether the blood glucose measurement value is less than a predetermined blood glucose hyperglycemia threshold; and
in response to the blood glucose measurement value being greater than the predetermined blood glucose hyperglycemia threshold, deliver a reserve dosage of the estimated carbo carbohydrate-compensation dosage of insulin.

30. The drug delivery device of claim 19, further comprising:
after delivery of the reserve dosage the estimated carbo carbohydrate-compensation dosage of insulin, determine whether a subsequent blood glucose measurement value is less than the predetermined blood glucose hyperglycemia threshold; and
in response to the blood glucose measurement value being greater than the predetermined blood glucose hyperglycemia threshold, increase the estimated carbohydrate-compensation dosage for future delivery by a predetermined percentage of the estimated carbohydrate-compensation dosage.
